(19) United States
(12) Patent Application Publication
(10) Pub. No.: US 2011/0201497 A1
(43) Pub. Date: Aug. 18, 2011

(54) Composition Comprising A Pesticide and an Alkoxylate of Iso-Heptadecylamine

(75) Inventors: Paul Klingelhoefer, Mannheim (DE); Sophie Vogel, Mannheim (DE); Kevin Huyghe, Mannheim (DE); Gerd Haderlein, Grunstadt (DE); Gerhard Schnabel, Elsenfeld (DE); Marc Nolte, Mannheim (DE); Richard Roger Evans, Limburgerhof (DE)

(73) Assignee: BASF SE, Ludwigshafen (DE)

(21) Appl. No.: 13/026,695

(22) Filed: Feb. 14, 2011

Related U.S. Application Data
(60) Provisional application No. 61/304,850, filed on Feb. 16, 2010.

(51) Int. Cl. A01N 57/20; C07C 213/04; A01C 1/06; C07C 211/07; A01P 13/00

(52) U.S. Cl. 504/100; 564/508; 564/463; 504/206; 504/127

(57) ABSTRACT

The present invention relates to a composition comprising a pesticide and an alkoxylate. Moreover, the invention relates to the alkoxylate, to a process for its preparation and to its use as adjuvant in pesticide-comprising spray mixtures. The invention furthermore relates to a method of controlling phytopathogenic fungi and/or undesired vegetation and/or undesired insect or mite attack and/or for regulating the growth of plants, wherein the composition is allowed to act on the respective pests, their environment or the plants to be protected from the respective pest, on the soil and/or on undesirable plants and/or the crop plants and/or their environment. Furthermore, the invention relates to seed comprising the composition.

The present invention relates to a composition comprising a pesticide and an alkoxylate. Moreover, the invention relates to the alkoxylate, to a process for its preparation and to its use as adjuvant in pesticide-comprising spray mixtures. The invention furthermore relates to a method of controlling phytopathogenic fungi and/or undesired vegetation and/or undesired insect or mite attack and/or for regulating the growth of plants, wherein the composition is allowed to act on the respective pests, their environment or the plants to be protected from the respective pest, on the soil and/or on undesirable plants and/or the crop plants and/or their environment. Furthermore, the invention relates to seed comprising the composition. The present invention comprises combinations of preferred features with other preferred features.

Alkoxylates and their use in agrochemical formulations as adjuvants are generally known:

WO 2009/004044 discloses a herbicidal composition comprising a phenoxy-acid herbicide and an alkoxylated alkylamine as adjuvant.

U.S. Pat. No. 5,668,085 discloses a herbicidal composition comprising an aqueous solution of glyphosate and surfactant. The surfactant may be an alkoxylated alkylamine, the alkyl group comprising 8 to 22 carbon atoms.

WO 2007/97614 discloses herbicidal agents comprising a herbicide and a surfactant, which, for example, may be an alkoxylated alkylamine.

Alkoxylated alkylamines, in particular commercially available ethoxylated tallow fatty amines (POEA), have important toxic properties (such as irritation of the skin and the eyes) and ecotoxic properties (such as high ecotoxicity to aquatic organisms such as algae and daphnias). Thus, for example, POEA (CAS No. 61791-26-2), which is frequently present in Roundup® herbicides as a wetter, is considered to be relatively toxic to aquatic organisms (Tsui and Chu, Chemosphere 2003, 52, 1189-1197).

It was therefore an object of the present invention to find an adjuvant which is well suited to herbicides such as glyphosate while being less toxic (especially lower toxicity to aquatic organisms). Furthermore, the adjuvant should make possible a storage-stable formulation of the pesticides.

The object was solved by a composition comprising a pesticide and an alkoxylate, wherein the alkoxylate is an amine alkoxylate (A)

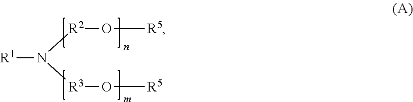

or a quaternized derivative (AQ)

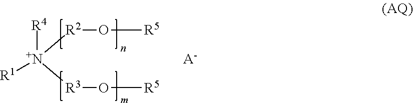

of the amine alkoxylate (A), where
R¹ is a branched aliphatic alkyl radical C₁₇H₃₅,
R², R³, and R⁷ independently of one another are ethylene, propylene, butylene or a mixture of these,
R⁴ is an H, —OH, —OR⁶, —[R⁷—O]ₚ—R⁵, C₁-C₆-alkyl or an oxygen anion,
R⁵ is an H, C₁-C₆-alkyl, C₂-C₆-alkenyl, C₂-C₆-alkynyl, —SO₃R^{a}, —P(O)OR^{b}OR^{c}, —CH₂CO₂R^{d} or —C(O)R^{e},
R⁶ is a C₁-C₆-alkyl, C₂-C₆-alkenyl or C₂-C₆-alkynyl,
R^{a} and R^{d} independently of one another are an H, inorganic or organic cations,
R^{b} and R^{c} independently of one another are an H, inorganic or organic cations, C₁-C₆-alkyl, C₂-C₆-alkenyl or C₂-C₆-alkynyl,
R^{e} is C₁-C₂₂-alkyl, C₂-C₂₂-alkenyl, C₂-C₂₂-alkynyl, C₆-C₂₂-aryl, C₇-C₂₂-alkylaryl,
n, m and p independently of one another are a value from 1 to 30, and
A- is an agriculturally acceptable anion, or, if R⁴ is an oxygen anion, A- is absent.

Preferably, the composition according to the invention comprises a pesticide and an alkoxylate, wherein the alkoxylate is an amine alkoxylate (A).

The radical R¹ is a branched aliphatic alkyl radical C₁₇H₃₅. Preferably, R¹ comprises a plurality of different branched aliphatic C₁₇H₃₅ alkyl radicals. The mean degree of branching of R¹ is in most cases 2.8 to 3.7, preferably 2.9 to 3.6, especially preferably 3.01 to 3.5, very especially preferably 3.05 to 3.4, and for example approximately 3.1.

In this context, the term “degree of branching” is defined in principle in the known manner as the number of methyl groups in a molecule of the alcohol minus 1. The mean degree of branching is the statistic mean of the degrees of branching of all molecules of a sample. In other words, the radical R¹ employed may take the form of a mixture of different alkyl groups C₁₇H₃₅. Accordingly, the alcohol R¹—OH, which is employed as starting material for the synthesis, may take the form of a mixture of different alcohols.

The mean degree of branching can be determined as follows by ¹H NMR spectroscopy: to this end, a sample of the alcohol R¹—OH is first subjected to derivatization with trichloroacetyl isocyanate (TAI). In this process, the alcohols are converted into the carbamic esters. The signals of the esterified primary alcohols are located at [symbol] =4.7 to 4.0 ppm, those of the esterified secondary alcohols (if present) at approximately 5 ppm, and water present in the sample reacts with TAI to give carbamic acid. All methyl, methylene and methyne protons are in the range of from 2.4 to 0.4 ppm. The signals <1 ppm are assigned to the methyl groups. The mean degree of branching (iso index) can be calculated from the spectrum thus obtained, as follows:

Iso Index=((F(CH₃)/3)/(F(CH₂—OH)/2))−1,

where F(CH₃) represents the signal area corresponding to the methyl protons and F(CH₂—OH) represents the signal area of the methylene protons in the CH₂—OH group.

Preferably, n has a value of from 1 to 20, especially preferably from 1 to 15. Preferably, m has a value of from 1 to 20, especially preferably from 1 to 15. Preferably, p has a value of from 1 to 30, especially preferably from 1 to 20. The values of n, m and o are normally average values as they mostly arise upon the alkoxylation with alkoxides. Therefore, n, m and o can not only be integers, but also all values between the integers.

Preferably, in the case of the amine alkoxylate (A), the total of n and m is 2 to 40 and in its quaternized derivative (AQ) the total of n, m and p is 3 to 80.

In the case of the amine alkoxylate (A) the total of n and m is especially preferably 3 to 30 and specifically 5 to 25. In a further especially preferred embodiment, the total of n and m is 6 to 9, in particular 6.5 to 8.5 and in particular 6.9 to 7.9. In a further especially preferred embodiment, the total of n and m is 11 to 40, in particular 12 to 30 and in particular 13.5 to 25.

In the case of the quaternized derivative (AQ) of the amine alkoxylate (A), the total of n, m and p is especially preferably 3 to 40 and specifically 5 to 25.

R², R³ and R⁷ are preferably independently of one another ethylene, ethylene and propylene, ethylene and butylene, or ethylene, propylene and butylene. In a further preferred embodiment, R², R³ and R⁷ are propylene. In a further preferred embodiment, R², R³ and R⁷ are butylene. Especially preferably R², R³ and R⁷ independently of one another are ethylene, or ethylene and propylene. Very especially preferably, R², R³ and R⁷ are ethylene.

If R², R³ or R⁷ comprise a butylene radical, the latter may be present as a n-butylene, an isobutylene or a 2,3-butylene group, with n-butylene and isobutylene being preferred and n-butylene being most preferred.

R², R³ and R⁷ independently of one another may be a mixture of ethylene, propylene or butylene. In this context, for example one or all radicals R², R³ and R⁷ may comprise a mixture of these groups in each alkoxylate chain. Such mixtures can be linked to one another in any desired order, for example randomly or blockwise (such as one block ethylene and one block propylene). Also, it is possible for in each case one or more of the radicals R², R³ and R⁷ a complete alkoxylate chain to be composed of different alkylene groups. For example, R² and R³ may be composed of ethylene and R⁷ of propylene.

R⁴ is preferably an H, OH, C₁-C₄-alkyl or an oxygen anion, it is especially preferably an H, methyl, butyl or an oxygen anion. In a specifically preferred embodiment, R⁴ is a methyl. In a further specifically preferred embodiment, R⁴ is an oxygen anion. In a further specifically preferred embodiment, R⁴ is an H.

R⁵ is preferably an H or C₁-C₆-alkyl, especially preferably an H or methyl, in particular H.

R⁶ is preferably a C₁-C₆-alkyl, such as methyl.

R^{a} and R^{d} independently of one another are H, or inorganic or organic cations which can carry one or more positive charges. Examples of inorganic cations are cations of ammonium, Na⁺, K⁺, Mg⁺, Ca²⁺ or Zn²⁺. Examples of organic cations are methyl-ammonium, dimethylammonium, trimethylammonium, tetramethylammonium, (2-hydroxyethyl)ammonium, bis(2-hydroxyethyl)ammonium, tris(2-hydroxyethyl)-ammonium, tetra(2-hydroxyethyl)ammonium. Preferably R^{a} and R^{d} independently of one another are H or inorganic cations. If an inorganic or organic cation is present, then the associated anionic group would be formed on R⁶ by the corresponding functional group (for example —SO₃⁻, —P(O)O⁻O⁻ or —CH₂CO₂⁻).

R^{b} and R^{c} preferably independently of one another are H, inorganic or organic cations. Suitable inorganic or organic cations are those mentioned under R^{a}.

In a further embodiment, the radicals R^{a}, R^{b}, R^{c} and R^{d} in the quaternary derivative (AQ) independently of one another can be organic cations, the cationic group being the quaternary nitrogen cation of AQ itself. In this case, AQ could thus form a zwitter ion, the anionic group being formed on R⁶ in AQ by the corresponding functional group (for example —SO₃⁻, —P(O)O⁻O⁻ or —CH₂CO₂⁻), and the cationic group being formed by the quaternary nitrogen of AQ. In this zwitter-ion form of AQ, the presence of an agriculturally acceptable anion A⁻ is optional.

R^{e} is preferably C₁-C₁₂-alkyl, C₆-C₁₂-aryl or C₇-C₁₂-alkylaryl, especially preferably C₁-C₆-alkyl.

A⁻ is an agriculturally acceptable anion, as they are generally known to the skilled worker. Preferably, A⁻ is a halide (such as chloride or bromide), phosphate, sulfate or an anionic pesticide. Propionate, acetate or formate is also suitable as A⁻. Especially preferably, A⁻ is an anionic pesticide, such as a glyphosate anion or glufosinate anion. If R⁴ is an oxygen anion, an amine oxide is present. In this case, a further anion such as A⁻ is absent.

In the case of the amine alkoxylate (A), it is preferred that R² and R³ independently of one another are ethylene, ethylene and propylene, ethylene and butylene, or ethylene, propylene and butylene, and the total of n and m is 2 to 60, preferably 2 to 40, especially preferably 3 to 30 and in particular 5 to 25. In a further preferred embodiment, R² and R³ are ethylene, ethylene and propylene, ethylene and butylene, or ethylene, propylene and butylene and the total of n and m is 6 to 9, in particular 6.5 to 8.5 and in particular 6.9 to 7.9. In a further preferred embodiment, R² and R³ are ethylene, ethylene and propylene, ethylene and butylene, or ethylene, propylene and butylene and the total of n and m is 11 to 40, in particular 12 to 30 and in particular 13.5 to 25. In an especially preferred embodiment, R¹ and R² are ethylene, ethylene and propylene, ethylene and butylene, or ethylene, propylene and butylene and the total of n and m is 6 to 14, in particular 8 to 12 and in particular 9 to 11.

In the case of the amine alkoxylate (A), it is especially preferred that R² and R³ are ethylene, and the total of n and m is 2 to 60, preferably 2 to 40, especially preferably 3 to 30, and in particular 5 to 25. In a further especially preferred embodiment, R² and R³ are ethylene and the total of n and m is 6 to 9, in particular 6.5 to 8.5 and in particular 6.9 to 7.9. In a further especially preferred embodiment, R² and R³ are ethylene and the total of n and m is 11 to 40, in particular 12 to 30 and in particular 13.5 to 25.

Compounds (A) and (AQ) may be present as mixtures of stereoisomers or as isolated stereoisomers. Tautomers and betaines are likewise comprised by the structures (A) and (AQ).

The composition according to the invention will, in most cases, comprise from 0.1 to 90% by weight of the alkoxylate, preferably from 1 to 50% by weight and in particular from 3 to 30% by weight.

The term pesticide refers to at least one active substance selected from the group of the fungicides, insecticides, nematicides, herbicides, safeners, molluscicides, rodenticides and/or growth regulators. Preferred pesticides are fungicides, insecticides, herbicides and growth regulators. Especially preferred pesticides are herbicides and growth regulators. Mixtures of pesticides from two or more of the above-mentioned classes may also be used. The skilled worker is familiar with such pesticides, which can be found, for example, in Pesticide Manual, 14th Ed. (2006), The British Crop Protection Council, London. Suitable pesticides are:

### A) Strobilurins:

azoxystrobin, dimoxystrobin, coumoxystrobin, coumethoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, methyl 2-[2-(2,5-dimethylphenyloxymethyl)phenyl]-3-methoxyacrylate, 2-(2-(3-(2,6-di-chlorophenyl)-1-methylallylideneaminooxymethyl)phenyl)-2-methoxyimino-N-methylacetamide;

### B) Carboxamides:

carboxanilides: benalaxyl, benalaxyl-M, benodanil, bixafen, boscalid, carboxin, fenfuram, fenhexamid, flutolanil, furametpyr, isopyrazam, isotianil, kiralaxyl, mepronil, metalaxyl, metalaxyl-M (mefenoxam), ofurace, oxadixyl, oxycarboxin, penflufen (N-(2-(1,3-dimethylbutyl)phenyl)-1,3-dimethyl-5-fluoro-1H-pyrazole-4-carboxamide), penthiopyrad, sedaxane, tecloftalam, thifluzamide, tiadinil, 2-amino-4-methylthiazole-5-carboxanilide, N-(3′,4′,5′-trifluorobiphenyl-2-yl)-3-difluoromethyl-1-methyl-1H-pyrazole-4-carboxamide, N-(4′-trifluoromethylthiobiphenyl-2-yl)-3-difluoromethyl-1-methyl-1H-pyrazole-4-carboxamide, N-(2-(1,3,3-trimethylbutyl)-phenyl)-1,3-dimethyl-5-fluoro-1H-pyrazole-4-carboxamide;
carboxylic acid morpholides: dimethomorph, flumorph, pyrimorph;
benzamides: flumetover, fluopicolide, fluopyram, zoxamid;
other carboxamides: carpropamid, diclocymet, mandipropamid, oxytetracyclin, silthiofam, N-(6-methoxypyridin-3-yl)cyclopropanecarboxamide;

### C) Azoles:

triazoles: azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole;
imidazoles: cyazofamid, imazalil, imazalil sulfate, pefurazoate, prochloraz, triflumizole;
benzimidazoles: benomyl, carbendazim, fuberidazole, thiabendazole;
others: ethaboxam, etridiazole, hymexazole, 2-(4-chlorophenyl)-N-[4-(3,4-dimethoxy-phenypisoxazol-5-yl]-2-prop-2-ynyloxyacetamide;

### D) Nitrogenous Heterocyclyl Compounds

pyridines: fluazinam, pyrifenox, 3-[5-(4-chlorophenyl)-2,3-dimethylisoxazolidin-3-yl]-pyridine, 3-[5-(4-methylphenyl)-2,3-dimethylisoxazolidin-3-yl]pyridine;
pyrimidines: bupirimate, cyprodinil, diflumetorim, fenarimol, ferimzone, mepanipyrim, nitrapyrin, nuarimol, pyrimethanil;
piperazines: triforine;
pyrroles: fludioxonil, fenpiclonil;
morpholines: aldimorph, dodemorph, dodemorph acetate, fenpropimorph, tridemorph;
piperidines: fenpropidin;
dicarboximides: fluorimid, iprodione, procymidone, vinclozolin;
nonaromatic 5-membered heterocyclic rings: famoxadon, fenamidon, flutianil, octhilinone, probenazole, S-ally 5-amino-2-isopropyl-3-oxo-4-orthotolyl-2,3-dihydro-1
others: acibenzolar-5-methyl, amisulbrom, anilazin, blasticidin-S, captafol, captan, quinomethionate, dazomet, debacarb, diclomezine, difenzoquat, difenzoquat methylsulfate, fenoxanil, folpet, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxypyrimidin-2-yl)-2-methyl-1H-benzimidazole, 5-chloro-7-(4-methylpiperidin-1-yl)-6-(2,4,6-trifluorophenyl)-[1,2,4]-triazolo[1,5-a]pyrimidine, 5-ethyl-6-octyl-[1,2,4]-triazolo[1,5-a]pyrimidin-7-ylamine;

### E) Carbamates and Dithiocarbamates

thio- and dithiocarbamates: ferbam, mancozeb, maneb, metam, methasulphocarb, metiram, propineb, thiram, zineb, ziram;
carbamates: diethofencarb, benthiavalicarb, iprovalicarb, propamocarb, propamocarb hydrochloride, valiphenal, (4-fluorophenyl) N-(1-(1-(4-cyanophenyl)ethanesulfonyl)-but-2-yl)carbamate;

### F) Other Fungicides

guanidines: dodine, dodine free base, guazatine, guazatine acetate, iminoctadine, iminoctadine triacetate, iminoctadine tris(albesilate);
antibiotics: kasugamycin, kasugamycin hydrochloride hydrate, polyoxins, streptomycin, validamycin A;
nitrophenyl derivatives: binapacryl, dicloran, dinobuton, dinocap, nitrothal-isopropyl, tecnazene;
organometallic compounds: fentin salts such as, for example, fentin acetate, fentin chloride, fentin hydroxide;
sulfurous heterocyclyl compounds: dithianon, isoprothiolane;
organophosphorus compounds: edifenphos, fosetyl, fosetyl-aluminum, iprobenfos, phosphorous acid and its salts, pyrazophos, tolclofos-methyl;
organochlorine compounds: chlorthalonil, dichlofluanid, dichlorphen, flusulfamide, hexachlorobenzene, pencycuron, pentachlorophenol and its salts, phthalide, quintozene, thiophanate-methyl, tolylfluanid, N-(4-chloro-2-nitrophenyl)-N-ethyl-4-methylbenzenesulfonamide;
inorganic active substances: phosphorous acid and its salts, Bordeaux mixture, copper salts such as, for example, copper acetate, copper hydroxide, copper oxychloride, basic copper sulfate, sulfur;
biological products for controlling fungi, plant strengthening products: Bacillus subtilis strain NRRL No. B-21661 (for example the products RHAPSODY®, SERENADE® MAX and SERENADE® ASO from AgraQuest, Inc., USA.), Bacillus pumilus strain NRRL No. B-30087 (for example SONATA® and BALLAD® Plus from AgraQuest, Inc., USA), Ulocladium oudemansii (for example BOTRY-ZEN from BotriZen Ltd., New Zealand), chitosan (for example ARMOUR-ZEN from BotriZen Ltd., New Zealand).
others: biphenyl, bronopol, cyflufenamid, cymoxanil, diphenylamine, metrafenon, mildiomycin, oxine-copper, prohexadione-calcium, spiroxamin, tolylfluanid, N-(cyclo-propylmethoxyimino-(6-difluoromethoxy-2,3-difluorophenyl)methyl)-2-phenyl-acetamide, N′-(4-(4-chloro-3-trifluoromethylphenoxy)-2,5-dimethylphenyl)-N-ethyl-N-methylformamidine, N′-(4-(4-fluoro-3-trifluoromethylphenoxy)-2,5-dimethylphenyl)-N-ethyl-N-methylformamidine, N′-(2-methyl-5-trifluoromethyl-4-(3-trimethylsilanyl-propoxy)phenyl)-N-ethyl-N-methylformamidine, N′-(5-difluoromethyl-2-methyl-4-(3-tri-methylsilanylpropoxy)phenyl)-N-ethyl-N-methylformamidine, N-methyl-(1,2,3,4-tetra-hydronaphthalen-1-yl)-2-{1-[2-(5-methyl-3-trifluoromethylpyrazol-1-yl)acetyl]piperidin-4-yl}thiazole-4-carboxylate, N-methyl-(R)-1,2,3,4-tetrahydronaphthalen-1-yl2-{1-[2-(5-methyl-3-trifluoromethylpyrazol-1-yl)acetyl]piperidin-4-yl}thiazole-4-carboxylate, 6-tert-butyl-8-fluoro-2,3-dimethylquinolin-4-ylacetate, 6-tert-butyl-8-fluoro-2,3-di-methylquinolin-4-yl methoxyacetate, N-methyl-2-{1-[2-(5-methyl-3-trifluoromethyl-1H-pyrazol-1-yl)acetyl]piperidin-4-yl}-N-[(1R)-1,2,3,4-tetrahydronaphthalen-1-yl]-4-thiazolecarboxamide;

### G) Growth Regulators

abscisic acid, amidochlor, ancymidole, 6-benzylaminopurine, brassinolide, butralin, chlormequat (chlormequat chloride), choline chloride, cyclanilid, daminozide, dikegulac, dimethipin, 2,6-dimethylpuridine, ethephon, flumetralin, flurprimidol, fluthiacet, forchlorfenuron, gibberellic acid, inabenfid, indole-3-acetic acid, maleic hydrazide, mefluidid, mepiquat (mepiquat chloride), metconazole, naphthaleneacetic acid, N-6-benzyladenine, paclobutrazole, prohexadione (prohexadione-calcium), prohydrojasmone, thidiazuron, triapenthenol, tributylphosphorotrithioate, 2,3,5-triiodo-benzoic acid, trinexapac-ethyl and uniconazole;

### H) Herbicides

acetamide: acetochlor, alachlor, butachlor, dimethachlor, dimethenamid, flufenacet, mefenacet, metolachior, metazachlor, napropamid, naproanilid, pethoxamid, pretilachlor, propachlor, thenylchior;
amino acid analogs: bilanafos, glyphosate, glufosinate, sulfosate;
aryloxyphenoxypropionates: clodinafop, cyhalofop-butyl, fenoxaprop, fluazifop, haloxyfop, metamifop, propaquizafop, quizalofop, quizalofop-P-tefuryl;
bipyridyls: diquat, paraquat;
carbamates and thiocarbamates: asulam, butylate, carbetamide, desmedipham, dimepiperat, eptam (EPTC), esprocarb, molinate, orbencarb, phenmedipham, prosulfocarb, pyributicarb, thiobencarb, triallate;
cyclohexanediones: butroxydim, clethodim, cycloxydim, profoxydim, sethoxydim, tepraloxydim, tralkoxydim;
dinitroanilines: benfluralin, ethalfluralin, oryzalin, pendimethalin, prodiamine, trifluralin;
diphenyl ethers: acifluorfen, aclonifen, bifenox, diclofop, ethoxyfen, fomesafen, lactofen, oyfluorfen;
hydroxybenzonitriles: bromoxynil, dichiobenil, ioxynil;
imidazolinones: imazamethabenz, imazamox, imazapic, imazapyr, imazaquin, imazethapyr;
phenoxyacetic acids: clomeprop, 2,4-dichlorophenoxyacetic acid (2,4-D), 2,4-DB, dichiorprop, MCPA, MCPA-thioethyl, MCPB, mecoprop;
pyrazines: chloridazon, flufenpyr-ethyl, fluthiacet, norfiurazon, pyridate;
pyridines: aminopyralid, clopyralid, diflufenican, dithiopyr, fluridone, fluoroxypyr, picloram, picolinafen, thiazopyr;
sulfonylureas: amidosulfuron, azimsulfuron, bensulfuron, chiorimuron-ethyl, chiorsulfuron, cinosulfuron, cyclosulfamuron, ethoxysulfuron, flazasulfuron, fluceto-sulfuron, flupyrsulfuron, foramsulfuron, halosulfuron, imazosulfuron, iodosulfuron, mesosulfuron, metsulfuron-methyl, nicosulfuron, oxasulfuron, primisulfuron, pro-sulfuron, pyrazosulfuron, rimsulfuron, sulfometuron, sulfosulfuron, thifensulfuron, tria-sulfuron, tribenuron, trifloxysulfuron, triflusulfuron, tritosulfuron, 1-((2-chloro-6-propyl-imidazo[1,2-b]pyridazin-3-yl)sulfonyl)-3-(4,6-dimethoxypyrimidin-2-yl)urea;
triazines: ametryne, atrazine, cyanazine, dimethametryne, ethiozine, hexazinone, metamitron, metribuzine, prometryne, simazine, terbuthylazine, terbutryne, triaziflam;
ureas: chlortoluron, daimuron, diuron, fluometuron, isoproturon, linuron, methabenz-thiazuron, tebuthiuron;
other acetolactate synthase inhibitors: bispyribac-sodium, cloransulam-methyl, diclosulam, florasulam, flucarbazone, flumetsulam, metosulam, orthosulfamuron, penoxsulam, propoxycarbazone, pyribambenz-propyl, pyribenzoxim, pyriftalide, pyriminobac-methyl, pyrimisulfan, pyrithiobac, pyroxasulfon, pyroxsulam;
others: amicarbazone, aminotriazole, anilofos, beflubutamid, benazolin, bencarbazone, benfluresate, benzofenap, bentazone, benzobicyclon, bromacil, bromobutide, butafenacil, butamifos, cafenstrole, carfentrazone, cinidon-ethlyl, chlorthal, cinmethylin, clomazone, cumyluron, cyprosulfamid, dicamba, difenzoquat, diflufenzopyr, Drechslera monoceras, endothal, ethofumesate, etobenzanid, fentrazamide, flumiclorac-pentyl, flumioxazin, flupoxam, fluorochloridon, flurtamon, indanofan, isoxaben, isoxaflutol, lenacil, propanil, propyzamide, quinclorac, quinmerac, mesotrione, methylarsenic acid, naptalam, oxadiargyl, oxadiazone, oxaziclomefon, pentoxazone, pinoxaden, pyraclonil, pyraflufen-ethyl, pyrasulfotol, pyrazoxyfen, pyrazolynate, quinoclamin, saflufenacil, sulcotrione, sulfentrazone, terbacil, tefuryltrione, tembotrione, thiencarbazone, topramezone, 4-hydroxy-3-[2-(2-methoxyethoxymethyl)-6-trifluoromethylpyridin-3-carbonyl]bicyclo[3.2.1]oct-3-en-2-one,
ethyl (3-[2-chloro-4-fluoro-5-(3-methyl-2,6-dioxo-4-trifluoromethyl-3,6-dihydro-2H-pyrimidin-1-yl)phenoxy]pyridin-2-yloxy)acetate, methyl 6-amino-5-chloro-2-cyclo-propylpyrimidine-4-carboxylate, 6-chloro-3-(2-cyclopropyl-6-methylphenoxy)-pyridazin-4-ol, 4-amino-3-chloro-6-(4-chlorophenyl)-5-fluoropyridin-2-carboxylic acid, methyl 4-amino-3-chloro-6-(4-chloro-2-fluoro-3-methoxyphenyl)pyridin-2-carboxylate and methyl 4-amino-3-chloro-6-(4-chloro-3-dimethylamino-2-fluorophenyl)pyridin-2-carboxylate;

### I) Insecticides

organo(thio)phosphates: acephate, azamethiphos, azinphos-methyl, chlorpyrifos, chlorpyrifos-methyl, chlorfenvinphos, diazinon, dichlorvos, dicrotophos, dimethoat, disulfoton, ethion, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, suiprophos, tetrachlorvinphos, terbufos, triazophos, trichlorfon;
carbamates: alanycarb, aldicarb, bendiocarb, benfuracarb, carbaryl, carbofuran, carbosulfan, fenoxycarb, furathiocarb, methiocarb, methomyl, oxamyl, pirimicarb, propoxur, thiodicarb, triazamate;
pyrethroids: allethrin, bifenthrin, cyfluthrin, cyhalothrin, cyphenothrin, cypermethrin, alpha-cypermethrin, beta-cypermethrin, zeta-cypermethrin, deltamethrin, esfenvalerate, etofenprox, fenpropathrin, fenvalerate, imiprothrin, lambda-cyhalo-thrin, permethrin, prallethrin, pyrethrin I and II, resmethrin, silafluofen, tau-fluvalinate, tefluthrin, tetramethrin, tralomethrin, transfluthrin, profluthrin, dimefluthrin,
insect growth inhibitors: a) chitin synthesis inhibitors: benzoylureas: chlorfluazuron, cyramazin, diflubenzuron, flucycloxuron, flufenoxuron, hexaflumuron, lufenuron, novaluron, teflubenzuron, triflumuron; buprofezin, diofenolan, hexythiazox, etoxazole, clofentazin; b) ecdysone antagonists: halofenozide, methoxyfenozide, tebufenozide, azadirachtin; c) juvenoids: pyriproxyfen, methoprene, fenoxycarb; d) lipid biosynthesis inhibitors: spirodiclofen, spiromesifen, spirotetramate;
nicotine receptor agonists/antagonists: clothianidin, dinotefuran, imidacloprid, thiamethoxam, nitenpyram, acetamiprid, thiacloprid, 1-(2-chlorothiazol-5-ylmethyl)-2-nitrimino-3,5-dimethyl-[1,3,5]triazinane;
GABA antagonists: endosulfan, ethiprole, fipronil, vaniliprole, pyrafluprole, pyriprole, N-5-amino-1-(2,6-dichloro-4-methylphenyl)-4-sulfinamoyl-1H-pyrazole-3-thiocarbox-amide;
macrocyclic lactones: abamectin, emamectin, milbemectin, lepimectin, spinosad, spinetoram;
mitochondrial electron transport chain inhibitor (METI) I acaricides: fenazaquin, pyridaben, tebufenpyrad, tolfenpyrad, flufenerim;
METI II and III substances: acequinocyl, fluacyprim, hydramethylnone;
decouplers: chlorfenapyr;
inhibitors of oxidative phosphorylation: cyhexatin, diafenthiuron, fenbutatin oxide, propargite;
insect ecdysis inhibitors: cryomazin;
mixed function oxidase’ inhibitors: piperonyl butoxide;
sodium channel blockers: indoxacarb, metaflumizon;
others: benclothiaz, bifenazate, cartap, flonicamid, pyridalyl, pymetrozin, sulfur, thiocyclam, flubendiamid, chlorantraniliprole, cyazypyr (HGW86); cyenopyrafen, flupyrazofos, cyflumetofen, amidoflumet, imicyafos, bistrifluoron and pyrifluquinazone.

Preferred pesticides comprise at least one pesticide with at least one H-acidic group (such as carboxylic acid group, phosphonic acid group, phosphinic acid group) or their anionic salts (for example mono-, di- or trisalts). These anionic salts of the pesticides with an H-acidic group are also suitable as anionic pesticides in group A⁻. Preferred pesticides with an H-acidic group are herbicides with an H-acidic group. Examples of herbicides with an H-acidic group are amino acid analogs (such as glyphosate or glufosinate) or imidazolinones (such as imazamethabenz, imazamox, imazapic, imazapyr, imazaquin, imazethapyr).

Particularly preferred pesticides with an H-acidic group are glyphosate and glufosinate. In a further preferred embodiment, pesticides with an H-acidic group are imidazolinones.

Especially preferably, the pesticide comprises a pesticide with an H-acidic group and a further pesticide. In a further embodiment, the pesticide comprises mixtures of at least two pesticides with an H-acidic group, and optionally further pesticides (such as at least one fungicide, herbicide, insecticide and/or safener, fungicides and/or herbicides being preferred).

In a further preferred embodiment, the pesticide comprises glyphosate (for example as the free acid, sodium salt, sesquisodium salt, potassium salt, dipotassium salt, ammonium salt, diammonium salt, dimethylammonium salt, trimesium salt or isopropylamine salt) or glufosinate (for example as the ammonium salt). Especially preferably, the pesticide comprises glyphosate (for example as the potassium salt, ammonium salt, isopropylamine salt). Especially preferably, the pesticide comprises glyphosate or glufosinate, and additionally a further herbicide. In a further preferred embodiment, the pesticide comprises glyphosate or glufosinate, and additionally a further pesticide (such as at least one fungicide, herbicide, insecticide and/or safener, fungicides and/or herbicides being preferred).

The compositions according to the invention can furthermore also comprise adjuvants conventionally used for agrochemical formulations, the choice of the adjuvants depending on the specific use form, the type of formulation or the active substance. Examples of suitable adjuvants are solvents, solid carriers, surface-active substances (such as surfactants, solubilizers, protective colloids, welters and tackifiers), organic and inorganic thickeners, bactericides, antifreeze agents, antifoams, optionally colorants and adhesives (for example for the treatment of seed) or conventional adjuvants for bait formulations (for example attractants, feedants, bittering substances).

Suitable solvents are water or organic solvents such as mineral oil fractions of medium to high boiling point such as kerosene and diesel oil, furthermore coal tar oils and oils of vegetable or animal origin, aliphatic, cyclic and aromatic hydrocarbons, for example paraffins, tetrahydronaphthalene, alkylated naphthalenes and their derivatives, alkylated benzenes and their derivatives, alcohols such as methanol, ethanol, propanol, butanol and cyclohexanol, glycols, ketones such as cyclohexanone, gamma-butyrolactone, dimethyl fatty acid amides, fatty acids and fatty acid esters, and strongly polar solvents, for example amines such as N-methylpyrrolidone. In principle, it is also possible to use solvent mixtures and mixtures of the abovementioned solvents and water.

Solid carriers are mineral earths such as silicas, silica gels, silicates, talc, kaolin, limestone, lime, chalk, bole, loess, clay, dolomite, diatomaceous earth, calcium and magnesium sulfate, magnesium oxide, ground synthetic materials, fertilizers such as ammonium sulfate, ammonium phosphate, ammonium nitrate, ureas and vegetable products such as cereal meal, tree bark meal, wood meal and nutshell meal, cellulose powders or other solid carriers.

Surface-active substances (adjuvants, wetters, tackifiers, dispersants or emulsifiers) which are suitable are the alkali metal, alkaline-earth metal, ammonium salts of aromatic sulfonic acids, for example of lignosulfonic acid (Borresperse® types, Borregaard, Norway), phenolsulfonic acid, naphthalenesulfonic acid (Morwet® types, Akzo Nobel, USA) and dibutylnaphthalenesulfonic acid (Nekal® types, BASF, Germany), and of fatty acids, alkyl- and alkylarylsulfonates, alkyl ether, lauryl ether and fatty alcohol sulfates, and salts of sulfated hexa-, hepta- and octadecanols and of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl polyglycol ethers, tributylphenyl polyglycol ether, alkylaryl polyether alcohols, isotridecyl alcohol, fatty alcohol/ethylene oxide condensates, ethoxylated castor oil, polyoxyethylene or polyoxypropylene alkyl ethers, lauryl alcohol polyglycol ether acetate, sorbitol esters, lignin-sulfite liquors and proteins, denatured proteins, polysaccharides (for example methylcellulose), hydrophobe-modified starches, polyvinyl alcohol (Mowiol® types, Clariant, Switzerland), polycarboxylates (Sokalan® types, BASF, Germany), polyalkoxylates, polyvinylamine (Lupamin® types, BASF, Germany), polyethyleneimine (Lupasol® types, BASF, Germany), polyvinylpyrrolidone and their copolymers.

The composition according to the invention may comprise from 0.1 to 40% by weight, preferably from 1 to 30 and in particular from 2 to 20% by weight of surface-active substances, the amount of the alkoxylate (A) and (AQ) not being taken into consideration.

Suitable thickeners are compounds which impart to the formulation a modified flow behavior, i.e. high viscosity at rest and low viscosity in the agitated state. Examples are polysaccharides, proteins (such as casein or gelatins), synthetic polymers, or inorganic layered minerals. Such thickeners are commercially available, for example Xanthan Gum (Kelzan®, CP Kelco, USA), Rhodopol® 23 (Rhodia, France) or Veegum® (R.T. Vanderbilt, USA) or Attaclay® (Engelhard Corp., NJ, USA). The thickener content in the formulation depends on the efficacy of the thickener. The skilled worker will choose such a content that the desired viscosity of the formulation is obtained. The content will amount to from 0.01 to 10% by weight in most cases.

Bactericides may be added in order to stabilize the composition. Examples of bactericides are those based on dichlorophene and benzyl alcohol hemiformal and also isothiazolinone derivatives such as alkylisothiazolinones and benzoisothiazolinones (Acticide® MBS from Thor Chemie). Examples of suitable antifreeze agents are ethylene glycol, propylene glycol, urea and glycerol. Examples of antifoams are silicone emulsions (such as, for example, Silikon® SRE, Wacker, Germany or Rhodorsil®, Rhodia, France), long-chain alcohols, fatty acids, salts of fatty acids, organofluorine compounds and mixtures of these.

The composition according to the invention can preferably be present in the form of an agrochemical formulation. Examples of such formulations and their preparation are:
i) Water-soluble concentrates (SL, LS): 10 parts by weight of the active substances are dissolved using 90 parts by weight of water or a water-soluble solvent. Alternatively, wetters or other adjuvants are added. Upon dilution in water, the active substance dissolves. This gives a composition with an active substance content of 10% by weight.
ii) Dispersible concentrates (DC): 20 parts by weight of the active substances are dissolved in 70 parts by weight of cyclohexanone with addition of 10 parts by weight of a dispersant, for example polyvinylpyrrolidone. Upon dilution in water, a dispersion is obtained. The active substance content amounts to 20% by weight
iii) Emulsifiable concentrates (EC): 15 parts by weight of the active substances are dissolved in 75 parts by weight of xylene with addition of calcium dodecylbenzene-sulfonate and castor oil ethoxylate (in each case 5 parts by weight). Upon dilution in water, an emulsion is obtained. The composition has an active substance content of 15% by weight.
iv) Emulsions (EW, EO, ES): 25 parts by weight of the active substances are dissolved in 35 parts by weight of xylene with addition of calcium dodecylbenzene-sulfonate and castor oil ethoxylate (in each case 5 parts by weight). Using an emulsifier (for example Ultra-Turrax), this mixture is placed into 30 parts by weight of water and made into a homogeneous emulsion. Upon dilution in water, an emulsion results. The composition has an active substance content of 25% by weight.
v) Suspensions (SC, OD, FS): 20 parts by weight of the active substances are comminuted with addition of 10 parts by weight of dispersants and wetters and 70 parts by weight of water or an organic solvent in a stirred-ball mill to give a finely divided active substance suspension. Upon dilution in water, a stable suspension of the active substance is obtained. The active substance content in the composition amounts to 20% by weight.
vi) Water-dispersible and water-soluble granules (WG, SG): 50 parts by weight of the active substances are ground finely with addition of 50 parts by weight of dispersants and wetters and prepared as water-dispersible or water-soluble granules by means of technical apparatuses (for example extrusion, spray tower, fluidized bed). Upon dilution in water, a stable dispersion or solution of the active substance is obtained. The composition has an active substance content of 50% by weight.
vii) Water-dispersible and water-soluble powders (WP, SP, SS, WS): 75 parts by weight of the active substances are ground in a rotor-stator mill with addition of 25 parts by weight of dispersants and wetters and also silica gel. Upon dilution in water, a stable dispersion or solution of the active substance is obtained. The active substance content of the composition amounts to 75% by weight.
viii) Gels (GF): in a ball mill, 20 parts by weight of the active substances, 10 parts by weight of dispersant, 1 part by weight of gelling agent and 70 parts by weight of water or an organic solvent are ground to give a fine suspension. Upon dilution with water, a stable suspension with an active substance content of 20% by weight is obtained.
ix) Dusts (DP, DS): 5 parts by weight of the active substances are ground finely and mixed intimately with 95 parts by weight of finely divided kaolin. This gives a dust with an active substance content of 5% by weight.
x) Granules (GR, FG, GG, MG): 0.5 part by weight of the active substances is ground finely and associated with 99.5 parts by weight of carriers. Conventional methods to this end are extrusion, spray-drying or the fluidized bed. This gives granules for direct application with an active substance content of 0.5% by weight.
xi) ULV solutions (UL): 10 parts by weight of the active substances are dissolved in 90 parts by weight of an organic solvent, for example xylene. This gives a composition to be applied directly with an active substance content of 10% by weight.

In general, the compositions comprise from 0.01 to 95% by weight, preferably from 0.1 to 90% by weight, of the pesticides.

The user will generally use the composition according to the invention for use in a premetering device, in a knapsack sprayer, in a spray tank or in a spraying aircraft. Here, the formulation is brought to the desired use concentration with water and/or buffer, optionally with addition of further auxiliaries, whereby the ready-to-use spray mixture (known as a tank mix) is obtained. Usually, 50 to 500 liters of the ready-to-use spray mixture are applied per hectare of utilizable agricultural area, preferably from 100 to 400 liters. In specific segments, these amounts can also be exceeded (for example fruit production) or undershot (for example aircraft application). The active substance concentrations in the ready-to-use preparations may be varied within substantial ranges. In general, they are between 0.0001 and 10%, preferably between 0.01 and 1%.

Oils of various types, wetters, drift-reducing agents, stickers, spreaders, adjuvants, fertilizers, plant-strengthening products, trace elements, herbicides, bactericides, fungicides and/or pesticides may be added to the active substances or to the preparations comprising them, optionally also to the tank mix, immediately prior to use. These products can be admixed to the compositions according to the invention in the weight ratio 1:100 to 100:1, preferably 1:10 to 10:1. Adjuvants which are suitable within this context are in particular: organic-modified polysiloxanes, for example Break Thru S 240®; alcohol alkoxylates, for example Atplus® 245, Atplus® MBA 1303, Plurafac® LF 300 and Lutensol® ON 30; EO/PO block polymers, for example Pluronic® RPE 2035 and Genapol® B; alcohol ethoxylates, for example Lutensol® XP 80; and sodium dioctyl sulfosuccinate, for example Leophen® RA.

Depending on the nature of the desired effect, the application rates of the active substance when used in plant protection are between 0.001 and 2.0 kg of active substance per ha, preferably between 0.005 and 2 kg per ha, especially preferably between 0.05 and 0.9 kg per ha, in particular between 0.1 and 0.75 kg per ha.

The present invention furthermore relates to a method of controlling phytopathogenic fungi and/or undesired vegetation and/or undesired insect or mite attack and/or for regulating the growth of plants, wherein the composition according to the invention is allowed to act on the respective pests, their environment or the plants to be protected from the respective pest, on the soil and/or on undesirable plants and/or the crop plants and/or their environment.

Examples of suitable crop plants are cereals, for example wheat, rye, barley, triticale, oats or rice; beet, for example sugar or fodder beet; pome fruit, stone fruit and soft fruit, for example apples, pears, plums, peaches, almonds, cherries, strawberries, raspberries, currants or gooseberries; legumes, for example beans, lentils, peas, lucerne or soybeans; oil crops, for example oilseed rape, mustard, olives, sunflowers, coconut, cacao, castor beans, oil palm, peanuts or soybeans; cucurbits, for example pumpkins/squash, cucumbers or melons; fiber crops, for example cotton, flax, hemp or jute; citrus fruit, for example oranges, lemons, grapefruit or tangerines; vegetable plants, for example spinach, lettuce, asparagus, cabbages, carrots, onions, tomatoes, potatoes, pumpkin/squash or capsicums; plants of the laurel family, for example avocados, cinnamon or camphor; energy crops and industrial feedstock crops, for example maize, soybeans, wheat, oilseed rape, sugar cane or oil palm; maize; tobacco; nuts; coffee; tea; bananas; wine (dessert grapes and grapes for vinification); hops; grass, for example turf; sweetleaf (Stevie rebaudania); rubber plants; ornamentals and forest plants, for example flowers, shrubs, deciduous trees and coniferous trees, and propagation material, for example seeds, and harvested produce of these plants.

The term crop plants also includes those plants which have been modified by breeding, mutagenesis or recombinant methods, including the biotechnological agricultural products which are on the market or in the process of being developed. Genetically modified plants are plants whose genetic material has been modified in a manner which does not occur under natural conditions by hybridizing, mutations or natural recombination (i.e. recombination of the genetic material). Here, one or more genes will, as a rule, be integrated into the genetic material of the plant in order to improve the plant's properties. Such recombinant modifications also comprise posttranslational modifications of proteins, oligo- or polypeptides, for example by means of glycosylation or binding polymers such as, for example, prenylated, acetylated or farnesylated residues or PEG residues.

Examples which may be mentioned are plants which, as the result of plant-breeding and recombinant measures, have acquired a tolerance for certain classes of herbicides, such as hydroxyphenylpyruvate dioxygenase (HPPD) inhibitors, acetolactate synthase (ALS) inhibitors such as, for example, sulfonylureas (EP-A 257 993, U.S. Pat. No. 5,013,659) or imidazolinones (for example U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073), enolpyruvylshikimate 3-phosphate synthase (EPSPS) inhibitors such as, for example, glyphosate (see, for example, WO 92/00377), glutamine synthetase (GS) inhibitors such as, for example, glufosinate (see, for example, EP-A 242 236, EP-A 242 246) or oxynil herbicides (see, for example, U.S. Pat. No. 5,559,024). For example, breeding and mutagenesis have given rise to Clearfield® oilseed rape (BASF SE, Germany), which features tolerance for imidazolinones, for example imazamox. With the aid of recombinant methods, crop plants such as soybeans, cotton, maize, beet and oilseed rape have been generated which are resistant to glyphosate or glufosinate, and these are available by the brand names RoundupReady® (glyphosate-resistant, Monsanto, U.S.A.) and Liberty Link® (glufosinate-resistant, Bayer CropScience, Germany).

Also comprised are plants which, with the aid of recombinant measures, produce one or more toxins, for example those from the bacterial strain Bacillus. Toxins which are produced by such genetically modified plants comprise, for example, insecticidal proteins of Bacillus spp., in particular from B. thuringiensis, such as the endotoxins Cry1Ab, Cry1Ac, Cry1F, Cry1Fa2, Cry2Ab, Cry3A, Cry3Bb1, Cry9c, Cry34Ab1 or Cry35Ab1; or vegetative insecticidal proteins (VIPs), for example VIP1, VIP2, VIP3, or VIP3A; insecticidal proteins from nematode-colonizing bacteria, for example Photorhabdus spp. or Xenorhabdus spp.; toxins from animal organisms, for example wasp, spider or scorpion toxins; fungal toxins, for example from Streptomycetes; plant lectins, for example from pea or barley; agglutinins; proteinase inhibitors, for example trypsin inhibitors, serine protease inhibitors, patatin, cystatin or papain inhibitors; ribosome-inactivating proteins (RIPs), for example ricin, maize RIP, abrin, luffin, saporin or bryodin; steroid-metabolizing enzymes, for example 3-hydroxysteroid oxidase, ecdysteroid IDP glycosyl transferase, cholesterol oxidase, ecdysone inhibitors or HMG CoA-reductase; ion channel blockers, for example inhibitors of sodium or calcium channels; juvenile hormone esterase; receptors for the diuretic hormone (helicokinin receptors); stilbene synthase, bibenzyl synthase, chitinases and glucanases. These toxins can also be produced, in the plants, in the form of pretoxins, hybrid proteins, truncated or otherwise modified proteins. Hybrid proteins are distinguished by a novel combination of different protein domains (see, for example, WO 2002/015701). Further examples of such toxins or genetically modified plants which produce these toxins are disclosed in EP-A 374 753, WO 93/07278, WO 95/34656, EP-A 427 529, EP-A 451 878, WO 03/18810 and WO 03/52073. The methods for generating these genetically modified plants are known to the skilled worker and explained, for example, in the abovementioned publications. A large number of the abovementioned toxins impart to the plants which produce them a tolerance for pests from all taxonomic classes of the arthropods, in particular beetles (Coeleropta), dipterans (Diptera) and lepidopterans (Lepidoptera) and nematodes (Nematoda). Genetically modified plants which produce one or more genes which code for insecticidal toxins are described for example in the abovementioned publications and are in some cases commercially available such as, for example, YieldGard® (maize varieties which produce the toxin Cry1Ab), YieldGard® Plus (maize varieties which produce the toxins Cry1Ab and Cry3Bb1), Starlink® (maize varieties which produce the toxin Cry9c), Herculex® RW (maize varieties which produce the toxins Cry34Ab1, Cry35Ab1 and the enzyme phosphinothricin N-acetyltransferase [PAT]); NuCOTN® 33B (cotton varieties which produce the toxin Cry1Ac), Bollgard® I (cotton varieties which produce the toxin Cry1Ac), Bollgard® II (cotton varieties which produce the toxins Cry1Ac and Cry2Ab2); VIPCOT® (cotton varieties which produce a VIP toxin); NewLeaf® (potato varieties which produce the toxin Cry3A); Bt-Xtra®, NatureGard®, KnockOut®, BiteGard®, Protecta®, Bt11 (for example Agrisure® CB) and Bt176 from Syngenta Seeds SAS, France, (maize varieties which produce the toxin CrylAb and the PAT enzyme), MIR604 from Syngenta Seeds SAS, France (maize varieties which produce a modified version of the toxin Cry3A, see in this context WO 03/018810), MON 863 from Monsanto Europe S.A., Belgium (maize varieties which produce the toxin Cry3Bb1), IPC 531 from Monsanto Europe S.A., Belgium (cotton varieties which produce a modified version of the toxin Cry1Ac) and 1507 from Pioneer Overseas Corporation, Belgium (maize varieties which produce the toxin Cry1F and the PAT enzyme).

Also comprised are plants which, with the aid of recombinant measures, produce one or more proteins which bring about an increased resistance to, or ability to withstand, bacterial, viral or fungal pathogens such as, for example, so-called pathogenesis-related proteins (PR proteins, see EP-A 0 392 225), resistance proteins (for example potato varieties which produce two resistance genes against Phytophthora infestans from the Mexican wild potato Solanum bulbocastanum) or T4 lysozyme (for example potato varieties which, as the result of the production of this protein, are resistant to bacteria such as Erwinia amylvora).

Also comprised are plants whose productivity has been improved with the aid of recombinant methods, for example by increasing the yield potential (for example biomass, grain yield, starch content, oil content or protein content), the tolerance for drought, salt or other limiting environmental factors, or the resistance to pests and fungal, bacterial and viral pathogens.

Also comprised are plants whose constituents, in particular for improving human or animal nutrition, have been modified with the aid of recombinant methods, for example by oil plants producing health-promoting long-chain omega-3-fatty acids or monounsaturated omega-9-fatty acids (for example Nexera® oilseed rape, DOW Agro Sciences, Canada).

The present invention also relates to seed (such as seeds or other plant propagation materials) comprising the composition according to the invention. Plant propagation materials can be treated preventively with the composition according to the invention at the point of or even before sowing or at the point of or even before transplanting. For the treatment of seed, one will generally use water-soluble concentrates (LS), suspensions (FS), dusts (DS), water-dispersible and water-soluble powders (WS, SS), emulsions (ES), emulsifiable concentrates (EC) and gels (GF). These compositions can be applied to the propagation materials, in particular seed, in undiluted form or, preferably, in diluted form. Here, the composition in question can be diluted 2- to 10-fold, so that from 0.01 to 60% by weight, preferably from 0.1 to 40% by weight, of active substance is present in the compositions used for the seed dressing. The application may be effected before or during sowing. The treatment of plant propagation material, in particular the treatment of seed, is known to the skilled worker and carried out by dusting, coating, pelleting, dipping or soaking the plant propagation material, the treatment preferably being carried out by pelleting, coating and dusting or by in-furrow treatment so that, for example, untimely early germination of the seed is prevented. It is preferred to use suspensions for the treatment of seed. Usually, such compositions comprise from 1 to 800 g/l of active substance, from 1 to 200 g/l of surfactants, from 0 to 200 g/l of antifreeze agents, from 0 to 400 g/l of binders, from 0 to 200 g/l of colorants and solvent, preferably water.

The present invention furthermore relates to an alkoxylate, wherein the alkoxylate is an amine alkoxylate (A)

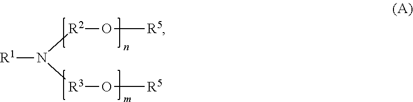

where
R¹ is a branched aliphatic alkyl radical C₁₇H₃₅,
R², R³, and R⁷ independently of one another are ethylene, propylene, butylene or a mixture of these,
R⁴ is an H, —OH, —OR⁶, —[R⁷—O]ₚ—R⁵, C₁-C₆-alkyl or an oxygen anion,
R⁵ is an H, C₁-C₆-alkyl, C₂-C₆-alkenyl, C₂-C₆-alkynyl, —SO₃R^{a}, —P(O)OR^{b}OR^{c}, —CH₂CO₂R^{d} or —C(O)R^{e},
R⁶ is a C₁-C₆-alkyl, C₂-C₆-alkenyl or C₂-C₆-alkynyl,
R^{a} and R^{d} independently of one another are an H, inorganic or organic cations,
R^{b} and R^{c} independently of one another are an H, inorganic or organic cations, C₁-C₆-alkyl, C₂-C₆-alkenyl or C₂-C₆-alkynyl,
R^{e} is C₁-C₂₂-alkyl, C₆-C₂₂-aryl, C₇-C₂₂-alkylaryl,
n, m and p independently of one another are a value from 1 to 30, and
A⁻ is an agriculturally acceptable anion, or, if R⁴ is an oxygen anion, A⁻ is absent.

Further preferred embodiments are as described above.

The present invention furthermore relates to an alkoxylate, wherein the alkoxylate is a quaternized derivative (AQ)

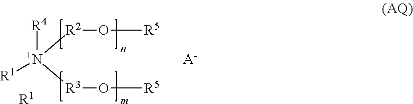

of the amine alkoxylate (A), where
R¹ is a branched aliphatic alkyl radical C₁₇H₃₅,
R², R³, and R⁷ independently of one another are ethylene, propylene, butylene or a mixture of these,
R⁴ is an H, —OH, —OR⁶, —[R⁷—O]ₚ—R⁵, C₁-C₆-alkyl or an oxygen anion,
R⁵ is an H, C₁-C₆-alkyl, C₂-C₆-alkenyl, C₂-C₆-alkynyl, —SO₃R^{a}, —P(O)OR^{b}OR^{c}, —CH₂CO₂R^{d} or —C(O)R^{e},
R⁶ is a C₁-C₆-alkyl, C₂-C₆-alkenyl or C₂-C₆-alkynyl,
R^{a} and R^{d} independently of one another are an H, inorganic or organic cations,
R^{b} and R^{c} independently of one another are an H, inorganic or organic cations, C₁-C₆-alkyl, C₂-C₆-alkenyl or C₂-C₆-alkynyl,
R^{e} is C₁-C₂₂-alkyl, C₆-C₂₂-aryl, C₇-C₂₂-alkylaryl,
n, m and p independently of one another are a value from 1 to 30, and
A⁻ is an agriculturally acceptable anion, or, if R⁴ is an oxygen anion, A⁻ is absent. Here, A⁻ is preferably a halide (such as chloride or bromide), phosphate, sulfate or an inorganic pesticide. A⁻ is especially preferably an inorganic pesticide, such as glyphosate anion or glufosinate anion. Further preferred embodiments are as described above.

The present invention furthermore relates to processes for the preparation of the amine alkoxylate (A) or a quaternized derivative (AQ) of the amine alkoxylate (A), comprising the alkoxylation of amines R¹—NH₂ with ethylene oxide, propylene oxide, butylene oxide or a mixture of these.

The present invention furthermore relates to the amine R¹—NH₂. It is an important intermediate for the preparation of the amine alkoxylate (A) or of a quaternized derivative (AQ) of the amine alkoxylate (A). Preferred embodiments of R¹ are as described above. The amines R¹—NH₂ can be prepared by reacting ammonia with alcohols R¹—OH. Suitable catalysts and reaction conditions are described in U.S. Pat. No. 5,808,158.

In principle, the alcohols R¹—OH can be synthesized by any method as long as they have in each case the degree of branching described. Alcohols R¹—OH may, for example, by obtained from a branch C₁₆-olefin by hydroformylation followed by hydration of the resulting aldehyde to give the alcohol. A person skilled in the art will, in principle, know how to carry out a hydroformylation and the subsequent hydrogenation, compare WO 2009/124979. The C₁₆-olefins employed for this purpose can be prepared by tetramerizing butene.

Preferably, the C₁₇-alcohol mixture can be prepared by
a) providing a hydrocarbon feedstock which comprises at least one olefin with 2 to 6 carbon atoms,
b) subjecting the hydrocarbon feedstock to oligomerization on a transition-metal-comprising catalyst,
c) subjecting the oligomerization product obtained in step b) to separation by distillation, whereby an olefin stream enriched in C₁₆-olefins is obtained,
d) subjecting the olefin stream enriched in C₁₆-olefins, which stream is obtained in step c), to hydroformylation by reacting it with carbon monoxide and hydrogen in the presence of a cobalt hydroformylation catalyst and subsequently subjecting the product to hydrogenation.

The details of the synthesis are generally known to the skilled worker, see, for example, WO 2009/124922 [step a) see page 8, line 27 to page 11, line 8; step b) see page 11, line 10 to page 13, line 41; step c) see page 14, line 1 to page 15, line 15; step d) see page 15, line 17 to page 17, line 26]. A particularly suitable alcohol R¹—OH can be prepared as described in WO 2009/124922 Example IA (page 29, line 5 to page 31, line 23).

The alkoxylation can be catalyzed by strong bases, such as alkali metal hydroxides and alkaline earth metal hydroxides, BrOnsted acids or Lewis acids, such as AlCl₃, BF₃. Catalysts such as hydrotalcite or DMC may be used for alcohol alkoxylates with a narrow distribution. The alkoxylation is preferably carried out at temperatures in the range of approximately 80 to 250° C., preferably approximately 100 to 220° C. The pressure is preferably between ambient pressure and 600 bar. If desired, the alkylene oxide may comprise an admixture of inert gas, for example of approximately 5 to 60%.

The quaternized derivative (AQ) of the amine alkoxylate (A) can be prepared in a further reaction step by quaternizing the amine alkoxylate (A). To introduce the radical R⁴ into the amine alkoxylate (A), the latter may be reacted for example with an alkylation reagent such as methyl chloride, dimethyl sulfate or butyl chloride. To introduce the one oxygen anion into the amine alkoxylate (A), the latter may be oxidized, for example by reacting the amino group with hydrogen peroxide, peracids (such as meta-chloroperbenzoic acid or peracetic acid) or peroxomonosulfuric acid.

The quaternized derivatives (AQ) where R⁴═H can be prepared by simple protonation of starting compounds of the structure (A). The quaternized derivatives (AQ) where R⁴═OH can be prepared by simple protonation of starting compounds (AQ) where R⁴=oxygen anion. Acids which are suitable for the protonation are organic acids (for example C₁- to C₂₀-carboxylic acids, in particular benzoic acid) or inorganic acids (for example hydrochloric acid, phosphoric acid or sulfuric acid). Others which are likewise suitable are H-acidic pesticides such as, for example, glyphosate-acid or glyphosate-monosalts. The protonation can be carried out in a separate synthesis, so that the quaternized derivative (AQ) can be isolated. It is also possible to carry out the protonation by mixing the starting compounds with one or more acids in the composition or in the spray mixture.

The present invention also relates to the use of the amine alkoxylate (A) or of a quaternized derivative (AQ) of the amine alkoxylate (A) as described above as auxiliary in pesticide-comprising spray mixtures. The auxiliary is preferably an activity-enhancing auxiliary. Such activity-enhancing auxiliaries are also referred to as adjuvants. They enhance or accelerate the activity of pesticides in comparison with the activity of the pesticide in the absence of the adjuvant.

The advantages of the invention are high stability of the formulation and of the spray mixture, little wind-caused drift in the case of spray applications, good adhesion of the formulation on the surface of the treated plants, increased solubility of the pesticides in the formulation, increased permeation of the pesticides into the plant and, as a result, more rapid and enhanced activity. An important advantage is the low toxicity of the novel alkoxylates, in particular the low aquatic toxicity. Another advantage is the low harmful effect toward useful plants, i.e. a low phytotoxic effect. A further advantage is the simple handling of these alkoxides since, for example, no gelling takes place upon their incorporation into formulations.

The examples which follow illustrate the invention without imposing any limitation.

### EXAMPLES

### Example 1

### Preparation of branched C₁₇-amine from C₁₇-alcohol

The branched C₁₇-alcohol (degree of branching 3.1) was prepared as in WO 2009/124922, example I (page 29, line 5 to page 31, line 23).

The C₁₇-alcohol (1500 g) and an alcohol amination catalyst (described in EP 696 572 A1; 6.7% by weight based on C₁₇-alcohol) were placed into the autoclave, and the autoclave was flushed with nitrogen and hydrogen. Then, 8000 g of ammonia were charged and warmed while stirring. After a reaction time of ten hours at 250° C. and H₂ pressure of 250 bar, the resulting C₁₇-amine was filtered and freed from water on a rotary evaporator. The amine content was 199.6 g of KOH/g and the water content was below 0.1% by weight.

### Example 2

### Alkoxylation of Branched C17-amine

First, 650 g (2.3 mol) of C₁₇-amine of example 1 (amine number 299 mg of KOH/g) were treated with 17.1 g of water. Then, 203 g (4.61 mol) of ethylene oxide (EO) were metered in over 12 h at 100° C., the metering being controlled by weight. Thereafter, stirring was continued for 6 h at 100° C. Subsequently, the mixture was dehydrated for 2 h at 90° C. in vacuo. This gave a yield of 855 g (=101% of theory) with an amine number of 152 mg of KOH/g (152 mg of KOH/g theory).

In the second step, 2.0 g of 50% strength KOH were added to 200 g (0.54 mol) of this precursor and the mixture was dehydrated for 2 h at 90° C. in vacuo. After flushing with nitrogen, 309 g (7.0 mol) of ethylene oxide were metered in over 6 h at 120° C., the metering being controlled by weight. Thereafter, stirring was continued for 6 h at 120° C. The product was degassed in vacuo and neutralized with a few drops of acetic acid. This gave iso-C₁₇-amine-15 EO in a yield of 513 g (101% of theory) as a pale brown fluid of low viscosity with an amine number of 60 mg of KOH/g (60 mg of KOH/g theory).

Iso-C₁₇-amine-10 EO and iso-C₁₇-amine-20 EO were prepared analogously in quantitative yield, with correspondingly less EO being employed in the second ethoxylation step.

### Example 3

### Glyphosate SL Against Winter Wheat and Soybean as Model Plant

To carry out greenhouse tests, the winter wheats (cv. Cubus) or soybeans (cv. Oxford) were sown or potted in loamy sand at a sowing depth of 1-2 cm. As soon as the plants had reached a height of 10 to 25 cm (i.e. approx. 10 to 21 days after sowing), the spray mixtures were applied to the plants in a spray cabinet.

A concentrated formulation comprising glyphosate isoproylammonium dissolved in water and amine alkoxylate of example 2 were diluted with fully demineralized water and were applied at a water application rate of 375 l/ha (140 g of glyphosate/ha and 300 g of amine alkoxylate/ha). The temperatures during the experimental period, which lasted for 3 to 4 weeks, were between 18 and 35° C. During this time, the test plants received optimal irrigation, the nutrient supply being effected via the irrigation water.

The herbicidal activity was assessed by scoring the treated plants in comparison with the untreated control plants (table 1). The assessment scale goes from 0% to 100% activity. 100% activity means complete dying at least of the aerial plant parts. In contrast, 0% activity means that no differences were observed between treated and untreated plants. The results in table 1 and 2 confirm the increased activity of the active substance by addition of the amine alkoxylate.

TABLE 1
Increased activity of glyphosate against model plant winter wheat

| | | Activity | | Activity |
|---|---|---|---|---|
| | | [%] | | [%] |
| | Glyphosate | after | Activity [%] | after |
| Amine alkoxylate | [g/ha] | 14 days | after 21 days | 28 days |
| —^{a)} | 140 | 64 | 73 | 73 |
| C_{16/18}-amine-15 EO^{a),b)} | 140 | 68 | 78 | 78 |
| iso-C₁₇-amine-10 EO | 140 | 95 | 100 | 100 |
| iso-C₁₇-amine-15 EO | 140 | 95 | 100 | 100 |
| iso-C₁₇-amine-20 EO | 140 | 93 | 100 | 100 |
| —^{a)} | 280 | 71 | 81 | 81 |
| C_{16/18}-amine-15 EO^{a),b)} | 280 | 86 | 100 | 100 |
| iso-C₁₇-amine-10 EO | 280 | 95 | 100 | 100 |
| iso-C₁₇-amine-15 EO | 280 | 95 | 100 | 100 |
| iso-C₁₇-amine-20 EO | 280 | 94 | 100 | 100 |

^{a)}comparative experiment, not in accordance with the invention.
^{b)}Genamine ® T-150, commercially available from Clariant, Germany.

TABLE 2
Increased activity of glyphosate against model plant soybean

| | | Activity | | Activity |
|---|---|---|---|---|
| | | [%] | | [%] |
| | Glyphosate | after | Activity [%] | after |
| Amine alkoxylate | [g/ha] | 14 days | after 21 days | 28 days |
| —^{a)} | 140 | 29 | 38 | 38 |
| C_{16/18}-amine-15 EO^{a),b)} | 140 | 44 | 50 | 50 |
| iso-C₁₇-amine-10 EO | 140 | 98 | 100 | 100 |
| iso-C₁₇-amine-15 EO | 140 | 98 | 100 | 100 |
| iso-C₁₇-amine-20 EO | 140 | 94 | 98 | 98 |
| —^{a)} | 280 | 36 | 40 | 40 |
| C_{16/18}-amine-15 EO^{a),b)} | 280 | 91 | 100 | 100 |
| iso-C₁₇-amine-10 EO | 280 | 98 | 100 | 100 |
| iso-C₁₇-amine-15 EO | 280 | 96 | 100 | 100 |
| iso-C₁₇-amine-20 EO | 280 | 95 | 100 | 100 |

^{a)}comparative experiment, not in accordance with the invention.
^{b)}Genamine ® T-150, commercially available from Clariant, Germany.

### Example 4

### Rainfastness

In each case four maize or winter wheat plants were treated with 560/ha of glyphosate isopropylammonium salt and 300 g/ha of amine alkoxylate. After 1.5 or 3 h after this treatment, the plants were irrigated by sprinkling with 100 l of water at a pressure of 3.33 bar and a velocity of 2.8 m/s for 20 min. The plants were subsequently placed into the greenhouse and scored after 14 and 21 days as in example 3. Genamine® T-150 (C_{16/18}-amine ethoxylate with 15 EO units, commercially available from Clariant) was employed for comparison purposes.

Tables 7 and 8 show that the glyphosate is less detached from the plant by the sprinkle irrigation, so that the efficacy remains high even after sprinkle irrigation.

TABLE 7
Efficacy [%] after 14 days

| | | | Sprinkle | Sprinkle |
|---|---|---|---|---|
| | | | irrigation | irrigation |
| | | Without | 1.5 h | 3 h |
| | | sprinkle | after | after |
| Amine alkoxylate | | irrigation | application | application |
| No application^{a)} | Maize | 0 | — | — |
| C_{16/18}-amine-15 | Maize | 86 | 45 | 55 |
| EO^{a),b)} | | | | |
| iso-C₁₇-amine-15 EO | Maize | 93 | 61 | 80 |
| No application^{a)} | Wheat | 0 | — | — |
| C_{16/18}-amine-15 | Wheat | 89 | 55 | 76 |
| EO^{a),b)} | | | | |
| iso-C₁₇-amine-15 EO | Wheat | 93 | 83 | 90 |

^{a)}comparative experiment, not in accordance with the invention.
^{b)}Genamine ® T-150, commercially available from Clariant, Germany.

TABLE 8
Efficacy [%] after 21 days

| | | | Sprinkle | Sprinkle |
|---|---|---|---|---|
| | | | irrigation | irrigation |
| | | Without | 1.5 h | 3 h |
| | | sprinkle | after | after |
| Amine alkoxylate | | irrigation | application | application |
| No application^{a)} | Maize | 0 | — | — |
| C_{16/18}-amine-15 | Maize | 98 | 63 | 69 |
| EO^{a),b)} | | | | |
| iso-C₁₇-amine-15 EO | Maize | 98 | 69 | 85 |
| No application^{a)} | Wheat | 0 | — | — |
| C_{16/18}-amine-15 | Wheat | 98 | 60 | 90 |
| EO^{a),b)} | | | | |
| iso-C₁₇-amine-15 EO | Wheat | 97 | 97 | 97 |

^{a)}comparative experiment, not in accordance with the invention.
^{b)}Genamine ® T-150, commercially available from Clariant, Germany.

1. A composition comprising a pesticide and an alkoxylate, wherein the alkoxylate is an amine alkoxylate (A) 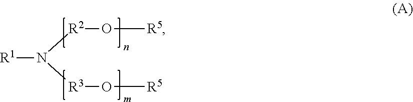
or a quaternized derivative (AQ) 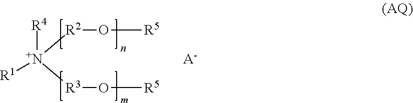
of the amine alkoxylate (A), wherein
R¹ is a branched aliphatic alkyl radical C₁₇H₃₅,
R², R³, and R⁷ independently of one another are ethylene, propylene, or butylene,
R⁴ is an H, —OH, —OR⁶, —[R⁷—O]ₚ—R⁵, C₁-C₆-alkyl or an oxygen anion,
R⁵ is an H, C₁-C₆-alkyl, C₂-C₆-alkenyl, C₂-C₆-alkynyl, —SO₃R^{a}, —P(O)OR^{b}OR^{c}, —CH₂CO₂R^{d} or —C(O)R^{e},
R⁶ is a C₁-C₆-alkyl, C₂-C₆-alkenyl or C₂-C₆-alkynyl,
R^{a} and R^{d} independently of one another are an H, an inorganic or an organic cation,
R^{b} and R^{c} independently of one another are an H, an inorganic or an organic cation, C₁-C₆-alkyl, C₂-C₆-alkenyl or C₂-C₆-alkynyl,
R^{e} is C₁-C₂₂-alkyl, C₂-C₂₂-alkenyl, C₂-C₂₂-alkynyl, C₆-C₂₂-aryl, or C₇-C₂₂-alkylaryl,
n, m and p independently of one another are a value from 1 to 30, and
A⁻ is an agriculturally acceptable anion, or, if R⁴ is an oxygen anion, A⁻ is absent.

2. The composition according to claim 1, wherein R², R³ and R⁷ independently of one another are ethylene, ethylene and propylene, ethylene and butylene, or ethylene, propylene and butylene.

3. The composition according to claim 1, wherein A⁻ is a halide, phosphate, sulfate or anionic pesticide.

4. The composition according to claim 1, wherein
R⁴ is H.

5. The composition according to claim 1, wherein, in the amine alkoxylate (A), the total of n and m is from 2 to 40, and in its quaternized derivative (AQ) the total of n, m and p is from 3 to 80.

6. The composition according to claim 1, wherein the mean degree of branching of R¹ is from 2.8 to 3.7.

7. The composition according to claim 1, wherein the pesticide comprises glyphosate or glufosinate.

8. The composition according to claim 1, wherein the pesticide comprises glyphosate or glufosinate, and additionally a further pesticide.

9. (canceled)

10. A process for the preparation of the amine alkoxylate (A) or a quaternized derivative (AQ) of the amine alkoxylate (A) according to claim 1, comprising alkoxylating an amine R¹—NH₂ with ethylene oxide, propylene oxide, butylene oxide or a mixture of these.

11. An amine R¹—NH₂, wherein R¹ is a branched aliphatic alkyl radical C₁₇H₃₅, wherein the mean degree of branching of R¹ is from 2.8 to 3.7.

12. A method of controlling phytopathogenic fungi and/or undesired vegetation and/or undesired insect or mite attack and/or for regulating the growth of plants, comprising allowing the composition according to claim 1 to act on the pests, their environment or the plants to be protected from the pest, on the soil and/or on undesirable plants and/or the crop plants and/or their environment.

13. A seed treated with the composition according to claim 1.

14. (canceled)
